(19) United States
(12) Patent Application Publication
(10) Pub. No.: US 2005/0281373 A1
(43) Pub. Date: Dec. 22, 2005

(54) Method for the automatic scaling verification of an image, in particular a patient image

(76) Inventors: Carlo Both, Veitsbronn (DE); Norbert Mukke, Herzogenaurach (DE); Artur Raczynski, Nurnberg (DE)

Correspondence Address: SCHIFF HARDIN, LLP;PATENT DEPARTMENT, 6600 SEARS TOWER, CHICAGO, IL, 60606-6473, US

(21) Appl. No.: 11/147,050

(22) Filed: Jun. 7, 2005

(30) Foreign Application Priority Data
Jun. 7, 2004 (DE) 10 2004 027 711.7

(51) Int. Cl. G01N 23/04; A61B 6/02

(52) U.S. Cl. 378/62

(57) ABSTRACT

In a method to reliably avoid erroneous length determination in the analysis of an image, in particular a digital patient image formed in the course of a medical imaging examination method, by automatic scaling or by the scaling verification of such an image, a shape segment inside the image range of the image is identified and selected by electronic image processing, at least one classification parameter determined according to predetermined criteria is assigned to the shape segment, at least one reference segment comparable to the classification parameter or parameters is selected from a reference database, the size of the shape segment is evaluated using the selected reference segment, and an evaluation quantity characterizing the result of this evaluation is formed.

### BACKGROUND OF THE INVENTION

1. Field of the Invention

The present invention relates to a method for the automatic scaling or for the scaling verification of an image, in particular a patient image formed in the course of a medical imaging examination.

2. Description of the Prior Art

Digital medical imaging examination methods are gaining constantly increasing importance in medicine. Medical imaging examination methods” encompass X-ray examination methods, examination methods based on nuclear magnetic resonance, ultrasound examination methods and photographic examination methods (for example endoscopy). These include both examination methods that produce static images and those which produce dynamic, i.e. moving images. Imaging examination methods that produce three-dimensional patient images are also well established (for example computed tomography and magnetic resonance tomography).

Image-assisted examination methods with similar requirements are furthermore employed in non-medical fields, for example in image-assisted industrial production and quality control processes.

The term “image” therefore refers in general to any type of digital data which allow a two-dimensional or three-dimensional representation of the spatial arrangement of entities or objects, and optionally allow representation of changes spatial arrangement as a function of time, or which allow the visual reproduction thereof. In particular, the term patient image refers to such an image that makes it possible to describe the spatial arrangement of body organs or objects in a patient's body. The term “image,” in particular “patient image,” particularly encompasses two-dimensional static or dynamic pixel data, especially photographic or video data, and static or dynamic voxel data, i.e. volume representations.

The analysis of a digital patient image generally is carried out on a computer with the aid of image processing application software. In such cases, it is often necessary to determine a distance between two marked image points, for example when establishing the size of a tumor or when preparing for an operative intervention.

To this end, conventionally, the number of image points lying between the marked positions in the vertical and horizontal image directions, or a corresponding length indicator contained in the image, is first recorded, and the mathematical distance between the marked positions in real three-dimensional space, i.e. in the patient's body, is then calculated therefrom. If only two-dimensional image data are available, instead of the distance in 3D space, its two-dimensional projection is computed. This distance is output in conventional length units (for example millimeters, inches, etc.).

This calculation works reliably only if correct information is available about the scaling or the pixel (voxel) resolution of the patient image, i.e. if knowledge is in principle available as to which geometrical separation in the body of the patient being imaged corresponds to the distance between two image points of the patient image. Usually in practice, an imaging facility, i.e. the examination device and the associated analysis device, are in fact generally calibrated so that the pixel resolution of a patient image is predetermined. Nevertheless, erroneous calibration information cannot entirely be ruled out. Typical sources of error are, in particular, typing mistakes during the manual entry of calibration information, a change in the imaging facility without appropriate recalibration being carried out, or intentional or inadvertent data manipulation.

If a distance inside a patient image is determined incorrectly because of erroneous calibration, this can have critical consequences for the patient's health or life, especially when an incorrect recommendation to operate is made on the basis of the erroneously determined distance.

### SUMMARY OF THE INVENTION

Against this background, it is an object of the present invention to provide a method that allows error-proof automatic scaling of an image, in particular a patient image, or alternatively automatic scaling verification to identify any miscalibration. The term scaling verification means checking the size ratios of entities or objects represented in an image.

This object is achieved according to the invention by a method, wherein a shape segment inside the image range of the image is first identified and selected by electronic image processing, particularly electronic shape recognition. In particular, a coherent group of image points that stands out from the surrounding image points with respect to color or brightness, or that is enclosed by an outline, is identified as a shape segment. In a patient image, bones, organs, blood vessels or even implants can be selected as a shape segment. The segmentation is optionally continued over a number of images in the same series, until suitable shape segments are found in an image.

At least one classification parameter is then assigned to the selected shape segment, and this is used to search for comparable reference segments in a reference database. If a comparable reference segment is found, this is selected and used as a reference for evaluating the size of the shape segment. An evaluation quantity that characterizes the result of the evaluation, i.e. reflects whether or how much the size of the shape segment corresponds to the size of the reference segment is formed for this purpose. In order to increase reliability, this comparison may be carried out on a number of, in particular at least three, different automatically selected segments.

The evaluation quantity is optionally used as a pure verification quantity, merely by displaying the evaluation quantity as an indicator of correct scaling or bad scaling of the patient image. Alternatively, the evaluation quantity is actively used to rescale the patient image when it found that the shape segment is significantly different from the reference segment in question.

One or more patient-specific parameters, exposure-specific parameters and/or geometric parameters characteristic of the selected shape segment preferably are employed as a classification parameter for a selected shape segment. Patient-specific parameters that may advantageously be employed for the classification includes the patient's age, the patient's sex, the patient's height, the patient's weight and/or a disease associated with the patient. Exposure-specific parameters that are suitable individually or in combination as classification parameters include the exposure projection on which the patient image is based, for example lateral, anterior-posterior, oblique, etc., and the body region being imaged, for example thorax, hip, abdomen, skull, extremities, etc. Suitable geometrical parameters are, in particular, the surface content and/or the circumference (i.e. the outline length) of the shape segment, the image position of the shape segment inside the image and/or the reference contour (for example approximately circular, elongated, etc.). It is preferable to use a predetermined set of several classification parameters that includes both patient-specific and exposure-specific and geometrical parameters. A combination of the parameters exposure projection, body region and image position of the shape segment is expedient, especially since under comparable exposure conditions it is very likely that shape segments corresponding to one another, for example the image of a particular vertebra, will always appear in the vicinity of the same image position. Further improved differentiation is possible, for example, if the patient's sex and/or the patient's height are added as further classification parameters.

By comparing the classification parameters with respectively corresponding parameters of the reference segments stored in the database, they are tested for a match with the shape segment. A reference segment is in this case selected when the classification parameters assigned to it correspond with the classification parameters of the shape segment, according to predetermined selection criteria. For example, a reference segment is selected only if the exposure-specific parameters assigned to the reference segment and the shape segment are the same, and if the image positions assigned to the reference segment and the shape segment match within predetermined tolerances.

For evaluating the size of the shape segment, at least one geometrical parameter of the shape segment and the corresponding parameter of a selected reference segment are determined, and these parameters are compared with one another. Preferably, a number of geometrical parameters of the shape segment are determined and compared with respectively corresponding parameters of the selected reference segment, in order to improve the statistical redundancy of the size comparison.

Alternatively or in addition, a number of comparable reference segments for a chosen shape segment, from which an average value of a geometrical parameter is first determined and then in turn compared with the corresponding geometrical parameter of the shape segment for evaluating the size of the shape segment.

In a preferred embodiment of the method, a binary evaluation quantity is produced in the form of a warning signal. This warning signal is emitted whenever the size of the shape segment differs significantly, i.e. by more than a predetermined tolerance threshold, from the size of the selected reference segment or—if a number of reference segments are used for the comparison—the average size of the selected reference segments.

In an alternative embodiment of the method, a scale factor that indicates the size difference between the shape segment and the reference segment, or the selected reference segments, is formed as the evaluation quantity. In this case, it is expedient to rescale the image according to the scale factor and thereby to match the shape segment, with respect to its size, to the reference segments.

In order to reduce the data processing load associated with carrying out the method, it is preferable not to use the entire image range of the image for selecting a shape segment. Instead, an image region, i.e. a part of the entire image range, is selected first and then the shape segment is selected inside this image region. The selection of the position of an image region preferably is carried out according to a random algorithm.

The error reliability of the method preferably is increased by selecting a number of image regions at different positions inside the image range of the image, at least one shape segment respectively being selected inside each image region. This ensures that an erroneous evaluation quantity cannot be produced owing to a local individual anatomical difference of the patient's body, for example an abnormal bone growth in the region of a vertebra.

If a number of shape segments is determined for an image, then it is expedient first to evaluate the size of each shape segment individually, i.e. a single-segment evaluation quantity is initially formed for each shape segment, and a multi-segment mean evaluation quantity is subsequently determined from these single-segment evaluation quantities, and is employed for the scaling or scaling verification of the image.

### DESCRIPTION OF THE DRAWINGS

FIG. 1 schematically shows a patient image in which two image regions are selected as an example for explaining the present invention, a shape segment being in turn selected in each image region.

FIG. 2 shows a method for the scaling verification of a patient image, in particular the patient image according to FIG. 1, in a flowchart.

FIG. 3 shows an alternative embodiment of the method in a representation corresponding to FIG. 2, the patient image being automatically scaled.

### DESCRIPTION OF THE PREFERRED EMBODIMENTS

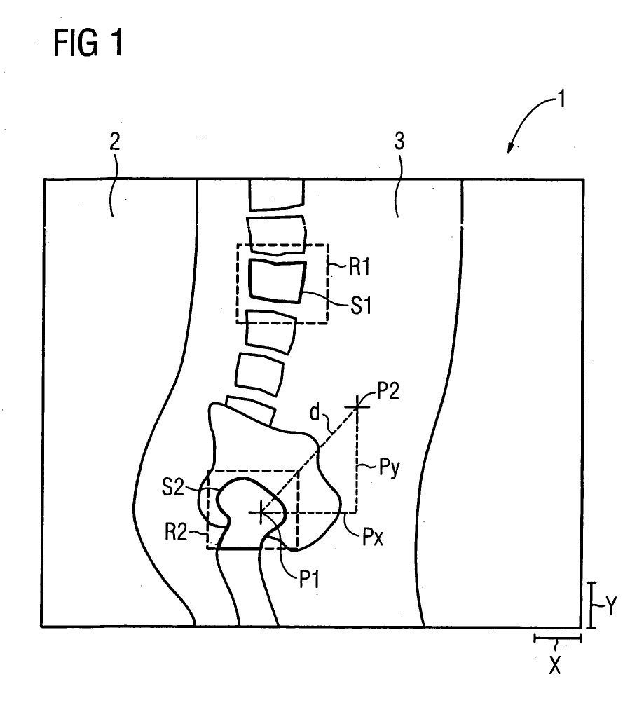

In order to illustrate the embodiment of the method respectively represented as simplified flowcharts in FIGS. 2 and 3, FIG. 1 schematically represents a two-dimensional patient image 1 as produced, for example, by a digital X-ray device. Such a digital patient image 1 includes a multiplicity of image points or pixels (not shown in detail) spatially arranged next to one another in a grid, each of which contains a color value or brightness value. The area covered by the image points (or the volume covered by the image points in the case of a three-dimensional patient image) is referred to as the image range 2.

The patient image 1 as represented shows a patient's body region 3 (in the example represented, the hip region in lateral projection).

The patient image 1 is assigned a horizontal scaling parameter X and a vertical scaling parameter Y. Each scaling parameter X, Y indicates the imaging scale of the patient image 1 in the corresponding space direction, in units of mm/pixel. In other words, the scaling parameters X, Y indicate the spatial distance in the patient's body, respectively in the horizontal and vertical directions, which corresponds to the distance between two horizontally or vertically adjacent image points. If this scaling X, Y is adjusted correctly, then the distance d between two image points P1 and P2 corresponds to a spatial distance d′ between two body positions of the patient, which (in a 2-dimensional projection) is given by
d′=\sqrt{(X·Pₓ)²+(Y·P_{y})²},
where Px denotes the number of image points lying between P1 and P2 in the horizontal direction, and Py denotes the number of image points lying between P1 and P2 in the vertical direction.

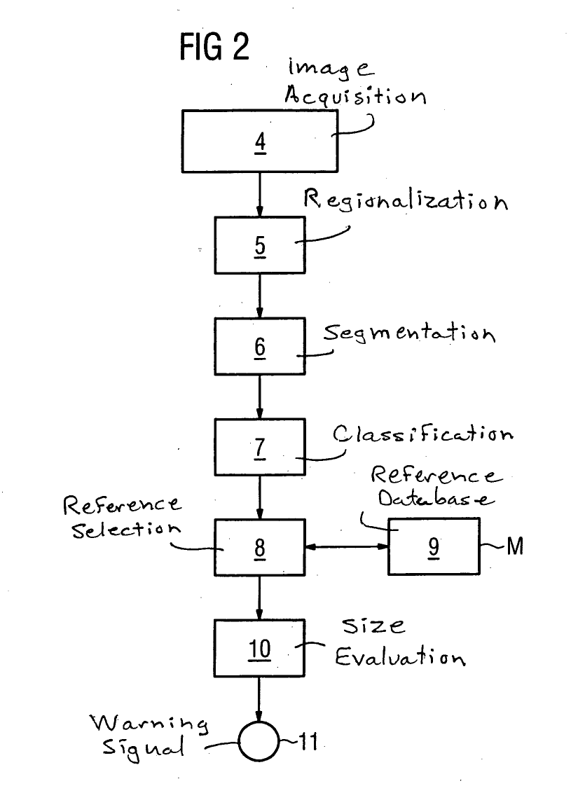

The validity of the scaling parameters X, Y of the patient image 1 is checked in the method indicated in a simplified flowchart in FIG. 2. The described method is intended to be carried out automatically inside an analysis station. A first method step involves the image acquisition 4. This generally comprises production of the patient image by means of an examination device, for example a digital X-ray device. Alternatively, the image acquisition 4 may be to load a pre-existing patient image 1 from an image archive or to digitize a patient image 1 available in analog form, for example by means of a scanner.

A subsequent method step involves regionalization 5 of the patient image 1. In this case, a number of image regions R1, R2 (indicated as rectangles with dashed borders in FIG. 1) are selected from the image range 2. The positions of the image regions R1, R2 inside the image range 2 are selected by using a random number algorithm.

This is followed, as a further method step, by segmentation 6 of each image region R1, R2. In this case, for each image region R1, R2, at least one image structure which stands out from the surrounding image points owing to a coherent outline or a color contrast is identified and selected by means of conventional electronic image processing methods as a shape segment S1 and S2, respectively. It is also possible to compile a color histogram over a particular image region, and to identify comparable image regions on the basis of this color histogram. In the representation according to FIG. 1, each image region R1 and R2 contains a selected shape segment S1 and S2, respectively. The image of a vertebra is selected as the image segment S1, and the image of the hip joint is selected as the image segment S2.

This is followed, in a further method step, by classification 7 of the selected shape segments S1 and S2. In this case, each shape segment S1 and S2 is assigned as classification parameters the exposure projection (in the example: lateral), the body region 3 being recorded (in the example, the hip), the respective image position of the shape segment S1, S2 inside the image range 2 and the patient's age, patient's height and patient's sex as classification parameters. Optionally, further geometrical parameters of the respective shape segment S1, S2, for example the length/height ratio, are determined and assigned as classification parameters.

A reference selection 8 is carried out with the aid of these classification parameters in a subsequent method step, in the course of which comparable reference segments M are searched for in a reference database 9. As reference segments M, the reference database 9 stores image structures such as those that typically occur in a patient image 1, in particular bones or bone parts, blood vessels or organs in various projections. The reference segments M stored in the reference database 9 are likewise assigned associated classification parameters, so that every classification parameter of the shape segment S1, S2 can be compared with a corresponding classification parameter of each reference segment M.

In this context, a reference segment M is selected if its classification parameters meet predetermined selection criteria with respect to the classification parameters assigned to the shape segment S1, S2. For example, a reference segment M is selected only if the reference segment M and the shape segment S1, S2 match exactly with respect to the classification parameters exposure projection, body region and patient sex and, with respect to the image position, within a predetermined tolerance range (for example 10%) for the image height and the image width.

Using the selected reference segments M, a size evaluation 10 of the respective shape segment S1, S2 is carried out in a subsequent method step. To this end, for example, the surface content and the outline length of the relevant shape segment S1, S2 are determined and compared with the correspondingly determined parameters of the respectively selected reference segments M. If a number of reference segments M are selected for a shape segment S1, S2, it is preferable firstly to determine the mean surface content and the mean outline length over the selected reference segments, and then to carry out the size comparison with these mean values.

If both the surface content and the outline length of the shape segment S1 or S2 differ by more than 3% from the comparative quantities of the associated reference segments, then a warning signal is set as a single-segment evaluation quantity for the corresponding shape segment S1 or S2. If the warning signal is set for more than a predetermined percentage of the selected shape segments S1 and S2, then a warning signal is in turn set as a multi-segment evaluation quantity and is displayed 11—for example on a screen of the analysis station. Optionally further shape segments can be selected in critical cases, in order to improve the statistical redundancy.

Displaying the warning signal indicates to the doctor operating the analysis station that the size of the shape segments S1, S2 calculated on the basis of the predetermined scaling parameters X, Y differs significantly from the empirical values stored in the reference database 9, from which it can be concluded that the scaling of the patient image 1 is wrong. The method according to FIG. 2 is thus used for scaling verification.

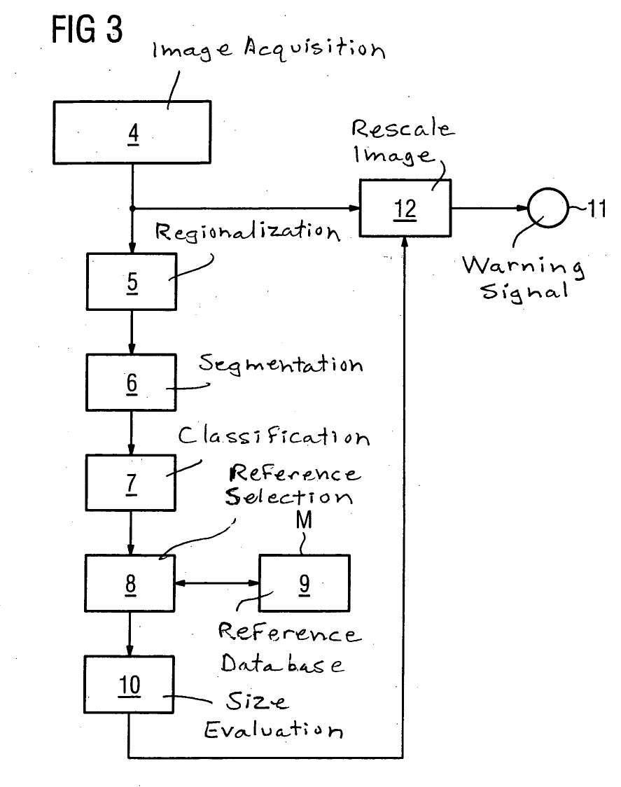

The embodiment of the method represented in FIG. 3 is the same as the procedure described above as regards the method steps image acquisition 4, regionalization 5, segmentation 6, classification 7 and reference selection 8. In the course of the size evaluation 10, however, the difference is that a scale factor showing the size difference between the shape segment S1, S2 and the reference segment M compared therewith, or the mean values of the selected reference segments M, is output as the evaluation quantity for each selected shape segment S1, S2. If the size evaluation is carried out by comparing the surface content and the outline length, then a scale factor suitable as an evaluation quantity may for example be determined with the aid of the formula

$$\sqrt{\frac{{A}_{S}}{{A}_{M}}} + \frac{1}{4} \cdot \frac{{1}_{S}}{{1}_{M}} .$$

Here, A_{S} and I_{S} stand for the surface content and the outline length of the respective shape segment S1, S2. A_{M} and I_{M} accordingly stand for the average surface content and the average outline length of the associated reference segments M.

From these single-segment scale factors, the average value is then formed as a multi-segment evaluation quantity. The scale factor determined in this way is employed in a subsequent method step to rescale 12 the patient image 1, with the previous scaling parameters X and Y of the patient image 1 being multiplied by the scale factor.

If the described method is carried out just after the patient image 1 is produced, in particular directly by the imaging facility, then real-time identification of wrongly scaled images can avoid unnecessary loading of a data network and an archive memory due to transmission and storage of these erroneous image data.

The data traffic in a data network can likewise be reduced if the segmentation 6 and the classification 7 are completed by the facility, and only those classification parameters which have been determined are sent to the reference database 9 for comparison.

Although modifications and changes may be suggested by those skilled in the art, it is the intention of the inventors to embody within the patent warranted hereon all changes and modifications as reasonably and properly come within the scope of their contribution to the art.

1. A method for automatically evaluating scaling of an image of a patient obtained in a medical imaging examination of the patient, comprising the steps of:
subjecting said patient image to electronic image processing and, in said electronic image processing, identifying a shape segment within an image range of said patient image;
assigning at least one classification parameter according to predetermined criteria to said shape segment;
from a reference database containing a plurality of reference segments, with at lease one classification parameter respectively assigned thereto, selecting at least one of said reference segments having a classification parameter assigned thereto comparable to the classification parameter assigned to said shape segment;
evaluating a size of said shape segment using said selected reference segment; and
from the evaluation of the size of said shape segment, generating an evaluation quantity indicative of whether said shape segment is correctly scaled in said patient image.

2. A method as claimed in claim 1 comprising employing at least one patient-specific parameter, selected from the group consisting of the age of the patient, the sex of the patient, the height of the patient, the weight of the patient, and a disease associated with the patient, as said classification parameter for said shape segment and said classification parameter for said reference segment.

3. A method as claimed in claim 1 comprising employing an exposure-specific parameter, selected from the group consisting of an exposure projection used to produce said patient image, and a body region of the patient shown in the patient image, as said classification parameter for said shape segment and said classification parameter for said reference segment.

4. A method as claimed in claim 1 comprising employing at least one geometrical parameter, selected from the group consisting of the surface content of said shape segment, the length of an outline of said shape segment, a position of said shape segment within said image range, and a contour of said shape segment, as said classification parameter for said shape segment, and employing a geometrical parameter, selected from the group consisting of the surface content of said reference segment, the length of an outline of said reference segment, a position of said reference segment within said image range, and a contour of said reference segment, as said classification parameter for said reference segment.

5. A method as claimed in claim 1 comprising comparing said classification parameter for said shape segment with classification parameters for respective reference segments in said reference database, and selecting said reference segment having a classification parameter comparable to the classification parameter of the shape segment that produces a comparison result satisfying predetermined selection criteria.

6. A method as claimed in claim 1 wherein the step of evaluating the size of said shape segment comprises comparing at least one geometrical parameter, selected from the group consisting of surface content, outline length and maximum extent in a predetermined direction, of said shape segment with a corresponding geometrical parameter of the selected reference segment.

7. A method as claimed in claim 1 wherein the step of selecting at least one reference segment comprises selecting a plurality of reference segments, as selected segments, and wherein the step of evaluating the size of said shape segment comprises formulating an average of a geometrical parameter of each of said selected shape segments, selected from the group consisting of surface content, outline length, and maximum extent in the a predetermined direction, and comparing said average to a corresponding geometrical parameter of the shape segment.

8. A method as claimed in claim 7 comprising generating a warning signal if said geometrical parameter of said shape segment differs by more than a predetermined tolerance threshold from said average.

9. A method as claimed in claim 1 comprising generating a warning signal if the size of said shape segment differs by more than a predetermined threshold from the size of said selected reference segment.

10. A method as claimed in claim 1 comprising forming a scale factor indicative of a difference in size between said shape segment and said reference segment.

11. A method as claimed in claim 10 comprising re-scaling said patient image according to said scale factor.

12. A method as claimed in claim 1 comprising identifying said shape segment within a predetermined image region of said image range.

13. A method as claimed in claim 12 comprising determining at least one of a position and an extent of said image region within said image range according to a random algorithm.

14. A method as claimed in claim 12 comprising selecting a plurality of image regions within said image range, and selecting at least one shape segment inside each image region.

15. A method as claimed in claim 1 comprising identifying a plurality of shape segments within said image range, evaluating the size of each of said plurality of shape segments with respect to at least one of said reference segments, and generating said evaluation quantity dependent on the evaluation of the respective sizes of all said shape segments.
